(19) United States
(12) Patent Application Publication
(10) Pub. No.: US 2026/0305643 A1
(43) Pub. Date: Oct. 8, 2026

(54) TEA COMPOSITIONS COMPRISING PARAXANTHINE AND METHODS OF PRODUCTION THEREOF

(71) Applicants: Evan Zhao, Cambridge, MA (US); Siyang Han, Cambridge, MA (US); Yutong Liu, Boston, MA (US)

(72) Inventors: Evan Zhao, Cambridge, MA (US); Siyang Han, Cambridge, MA (US); Yutong Liu, Boston, MA (US)

(21) Appl. No.: 19/640,823

(22) Filed: Apr. 7, 2026

Related U.S. Application Data
(60) Provisional application No. 63/784,445, filed on Apr. 7, 2025.

(51) Int. Cl. A23F 3/14; A23F 3/16; A23F 3/26; A23F 3/30; A23F 3/34; A23F 3/40; A61K 9/00; A61K 31/047; A61K 31/185; A61K 31/198; A61K 31/522; A61K 45/06

(52) U.S. Cl. CPC A23F 3/14; A23F 3/163; A23F 3/26; A23F 3/30; A23F 3/34; A23F 3/40; A61K 9/0095; A61K 31/047; A61K 31/185; A61K 31/198; A61K 31/522; A61K 45/06

(57) ABSTRACT

In certain aspects, the present disclosure provides tea compositions incorporating paraxanthine. Such compositions can comprise teas such as matcha, chrysanthemum, chocolate tea, and decaffeinated traditional varieties, offered in forms like leaves, powder, or beverages.

### RELATED APPLICATIONS

This application claims the benefit of U.S. Provisional Patent Application No. 63/784445, filed on Apr. 7, 2025, the contents of which are hereby incorporated by reference in their entirety.

### BACKGROUND

Tea is a widely consumed beverage known for its flavor, aroma, and potential health benefits. Traditional teas from the Camellia sinensis plant contain caffeine, which provides alertness and energy but can also cause side effects such as anxiety and insomnia in some individuals. Decaffeinated teas are available to address these issues, but they may not fully satisfy consumers due to changes in flavor or residual caffeine effects. Herbal teas, made from various plants like chrysanthemum and butterfly pea flowers, are naturally caffeine-free and offer unique flavors and benefits. However, they lack the stimulant properties of traditional teas, which some consumers desire. Accordingly, there is an unmet need for teas containing alternative stimulants to caffeine.

### BRIEF DESCRIPTION

In certain aspects, the present disclosure provides tea compositions comprising paraxanthine, or a salt or hydrate thereof, and a tea, wherein the tea composition is in a solid form or a liquid form. In some embodiments, the tea composition is in the form of tea leaves, tea powder, or a tea beverage.

In some embodiments, the tea comprises an herbal tea. In some embodiments, the tea comprises chrysanthemum tea. In some embodiments, the tea comprises Camellia sinensis. In some embodiments, the tea comprises matcha tea. In some embodiments, the tea is decaffeinated. In some embodiments, the tea is non-decaffeinated. In some embodiments, the tea comprises Theobroma caca (e.g., chocolate tea).

In some embodiments, the tea composition comprises at least one of creatine, Rhodiola rosea extract, Ashwagandha extract, taurine, lutein, zeaxanthin, L-theanine, or one or more B vitamins. In some embodiments, the tea composition comprises creatine. In some embodiments, the tea composition comprises Rhodiola rosea extract. In some embodiments, the tea composition comprises Ashwagandha extract. In some embodiments, the tea composition comprises taurine. In some embodiments, the tea composition comprises one or more B vitamins. In some embodiments, the tea composition comprises lutein. In some embodiments, the tea composition comprises zeaxanthin. In some embodiments, the tea composition comprises L-theanine.

In some embodiments, the tea composition comprises one or more sweeteners (e.g., natural or artificial sweeteners). In some embodiments, the sweeteners are selected from one or more of table sugar (sucrose), high-fructose corn syrup, honey, agave nectar, maple syrup, stevia, monk fruit extract, aspartame, sucralose, saccharin, acesulfame potassium (Ace-K), erythritol, xylitol, sorbitol, coconut sugar, date syrup, molasses, turbinado sugar, neotame, and allulose.

In some embodiments, the tea composition comprises one or more flavors (e.g., natural or artificial flavors). In some embodiments, the flavors comprise one or more of fruit flavors (e.g., flavors from or imitating lemon, lime, orange, grapefruit, peach, apple, pear, plum, cherry, watermelon, strawberry, raspberry, blueberry, blackberry), herbal flavors (e.g., mint, peppermint, basil, lavender, rosemary), nut (e.g., peanut, almond, hazelnut, tamarind, walnut, cashew, pecan), vanilla, ginger, cinnamon, turmeric, nutmeg, allspice, cardamom, cocoa or chocolate (e.g., cocoa powder, natural chocolate flavor), caramel, coconut, hibiscus, and rose. In some preferred embodiments, the flavors comprise natural peach flavor.

In some embodiments, the tea composition is in solid form (e.g., tea leaves or tea powder) and is preferably 0.005% to 10% by weight of the composition is paraxanthine. In some embodiments, tea composition comprises about 0.1% to about 5% paraxanthine by weight. In some embodiments, the tea composition is freeze-dried. In some embodiments, the tea composition is milled. In some embodiments, the tea composition is intended to be dissolved in water to form a tea beverage.

In some embodiments, the tea composition is in liquid form (e.g., a tea beverage), and the composition comprises 1 to 2000 mg/L paraxanthine. In some preferred embodiments, the tea composition comprises about 100 to about 700 mg/L paraxanthine.

In some preferred embodiments, the tea composition comprises about 0.2 g to about 2 g of paraxanthine per serving. In some especially preferred embodiments, the tea composition comprises about 0.2 g to about 0.5 g paraxanthine per serving.

In another aspect, the present disclosure provides methods of producing tea compositions disclosed herein. In some embodiments, the method comprises adding paraxanthine to a tea base comprising at least one of chrysanthemum tea, butterfly pea flower tea, Kenya purple leaf tea, decaffeinated tea from Camellia sinensis, tea from Theobroma caca, or matcha tea. In some embodiments, paraxanthine is ground with the tea base to achieve a uniform distribution.

### DESCRIPTION

Paraxanthine, a metabolite of caffeine, has been shown to provide similar benefits to caffeine with fewer side effects. It can enhance cognitive function and physical performance without causing significant anxiety or sleep disturbances. While paraxanthine has been studied and used in various dietary supplements, it has not yet been incorporated into tea compositions to provide the benefits of a stimulant without the drawbacks associated with caffeine. Tea compositions that incorporate paraxanthine offer a novel way for consumers to enjoy the taste and health benefits of tea while experiencing the desired stimulant effects without the negative side effects of caffeine.

Integration of paraxanthine into a product like tea that requires adequate taste and flavor profiles can be difficult. Paraxanthine can have a mildly bitter taste and flavoring that goes well with a tea product can require significantly more iteration than is routine. In addition, tea modalities can be hard to put into a satisfactory product and the introduction of a new ingredient like paraxanthine further complicates the use of e.g., spray dried or freeze dried tea compositions.

Provided herein are tea compositions comprising paraxanthine and methods of producing such compositions. In some embodiments, compositions comprise caffeinated, partially decaffeinated, and decaffeinated beverages and/or tea that further comprise paraxanthine, salts thereof, and/or hydrates thereof. Compositions can comprise decaffeinated traditional teas (i.e., where caffeine has been removed), herbal teas, and/or matcha tea (i.e., a powdered green tea, optionally decaffeinated). By providing such compositions, the invention addresses the needs of a wide range of consumers, from those who are sensitive to caffeine to those who seek enhanced performance and well-being through tea consumption.

The tea compositions comprising paraxanthine, salts and/or hydrates thereof disclosed herein offer the benefits of a stimulant without the negative side effects associated with caffeine. These compositions are designed to cater to consumers who are sensitive to caffeine or who prefer alternatives that provide energy and alertness without anxiety, restlessness, or sleep disturbances. The formula may be enhanced with additional ingredients, including creatine, Rhodiola rosea, Ashwagandha, taurine, B vitamins, lutein, zeaxanthin, L-theanine, natural flavors, artificial flavors, and sweeteners. Production methods involve grinding or soaking tea with paraxanthine. The compositions can deliver energy and alertness while minimizing anxiety and sleep disruption, appealing to health-conscious consumers seeking a caffeine-free stimulant option. The compositions can support visual health and protect retinal tissue. The compositions provide cognitive support.

### Benefits

The use of paraxanthine in tea compositions as described herein provides many benefits. For example, the compositions described herein provide stimulant effects without the side effects of caffeine, such as anxiety, jitteriness, and increased urination. The tea compositions are versatile across different tea types and forms, catering to both traditional tea drinkers and those preferring herbal options. The tea compositions are customizable with additional health-promoting ingredients, which improves energy, stress management, and taste. The methods of preparing the compositions also enable preservation of tea flavor and quality, with methods designed to maintain aroma and taste.

Paraxanthine has a shorter half-life (approximately 3.1 hours) compared to caffeine (approximately 4.1 hours), potentially allowing for better control over its effects and less disruption to sleep patterns, making it suitable for consumption later in the day or closer to bedtime.

### Definitions

Paraxanthine: 1,7-Dimethylxanthine (C₇H₈N₄O₂), a metabolite of caffeine known for its stimulant properties with reduced side effects compared to caffeine. As used herein, the term “paraxanthine” is inclusive of salts and hydrates thereof.
Tea compositions: Any composition derived from tea leaves or other plant materials traditionally used to make tea, including but not limited to, traditional teas from Camellia sinensis (such as green tea, black tea, oolong tea, white tea, matcha), herbal teas (such as chrysanthemum tea, butterfly pea flower tea, peppermint, lemongrass, or chicory root), chocolate tea (such as tea made from cacao bean husks, cacao nibs, or other parts of the cacao plant Theobroma caca, and combinations thereof.
Decaffeinated tea or decaf tea: Tea from which at least 90% of the caffeine has been removed. In some embodiments, decaffeinated tea contains less than 10 mg, less than 5 mg, or less than 2 mg caffeine per serving of tea, preferably less than 2 mg caffeine per serving.

As used herein in the specification, “a” or “an” may mean one or more. As used herein in the claim(s), when used in conjunction with the word “comprising”, the words “a” or “an” may mean one or more than one.

As used herein, the term “about” is defined as being close to, as understood by one of ordinary skill in the art. In one non-limiting embodiment, the term “about” is defined to be within 10%, preferably within 5%, more preferably within 1%, and most preferably within 0.5%.

### Composition Details

The tea compositions of the present disclosure comprise paraxanthine in amounts effective to provide the desired stimulant effects. The compositions can be in various forms, such as solid forms (e.g., tea leaves or tea powder) or liquid forms (e.g., tea beverages).

For solid forms (e.g., tea leaves or tea powder), paraxanthine is typically present in an amount from about 0.005% to about 10% by weight of the composition. In certain preferred embodiments, the composition comprises 0.1% to 5% paraxanthine by weight. For example, the composition can comprise about 0.1%, about 0.2%, about 0.3%, about 0.4%, about 0.5%, about 0.6%, about 0.7%, about 0.8%, about 0.9%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, or about 5% paraxanthine by weight.

For liquid forms (e.g., tea beverages), according to various embodiments, the concentration of paraxanthine is from about 1 to about 1000 mg/L. In some embodiments, the tea composition comprises about 10 mg/L, about 50 mg/L, about 100 mg/L, about 150 mg/L, about 200 mg/L, about 250 mg/L, about 300 mg/L, about 350 mg/L, about 400 mg/L, about 450 mg/L, about 500 mg/L, about 550 mg/L, about 600 mg/L, about 650 mg/L, about 700 mg/L, about 750 mg/L, about 800 mg/L, about 850 mg/L, about 900 mg/L, about 950 mg/L, or about 1000 mg/L paraxanthine. In some embodiments, the composition comprises 100 to 700 mg/L paraxanthine, e.g., about 100 mg/L, about 150 mg/L, about 200 mg/L, about 250 mg/L, about 300 mg/L, about 350 mg/L, about 400 mg/L, about 450 mg/L, about 500 mg/L, about 550 mg/L, about 600 mg/L, about 650 mg/L, or about 700 mg/L paraxanthine. In certain preferred embodiments, the tea compositions comprise about 200 to about 2000 mg/L paraxanthine. In some embodiments, the tea compositions comprise about 800 mg/L, about 900 mg/L, about 1000 mg/L, about 1100 mg/L, about 1200 mg/L, about 1300 mg/L, about 1400 mg/L, about 1500 mg/L, about 1600 mg/L, about 1700 mg/L, about 1800 mg/L, about 1900 mg/L, or about 2000 mg/L.

In some embodiments, the tea compositions comprise 0.1 to 5 g of paraxanthine per serving, e.g., 0.1 g, 0.2 g, 0.3 g, 0.4 g, 0.5 g, 0.6 g, 0.7 g, 0.8 g, 0.9 g, 1 gram, 1.5 g, 2 g, 2.5 g, 3 g, 3.5 g, 4 g, 4.5 g, or 5 g. In certain preferred embodiments, the tea compositions comprise about 0.2 g to about 2 g of paraxanthine per serving. In certain preferred embodiments, the tea compositions comprise about 0.2 g to about 0.5 g of paraxanthine per serving. In some preferred embodiments, the tea compositions comprise about 0.2 g of paraxanthine per serving. In other preferred embodiments, the tea compositions comprise about 0.5 g of paraxanthine per serving.

In some embodiments, the tea composition is in solid form and a serving is about 80 g, about 90 g, about 100 g, about 110 g, about 120 g, or about 130 g total weight.

In some embodiments, the tea composition is in liquid form and a serving is about 180 mL, about 200 mL, about 220 mL, about 250 mL, about 270 mL, or about 300 mL.

### Types of Tea Used

The invention encompasses a variety of tea types.
Decaffeinated traditional teas: Traditional teas from Camellia sinensis that have been decaffeinated to remove at least 90% of their caffeine content, including decaffeinated matcha.
Herbal teas: Teas made from plants other than Camellia sinensis, which are naturally caffeine-free, such as chrysanthemum, butterfly pea flower, peppermint, lemongrass, and chicory root. Herbal teas can include chocolate teas, e.g., teas made from cacao bean husks, cacao nibs, or other parts of the cacao plant Theobroma cacao.
Non-decaffeinated traditional teas: Traditional teas from Camellia sinensis that retain their natural caffeine content, including non-decaffeinated matcha.
Combinations: Tea compositions can comprise mixtures of traditional teas (decaffeinated or not) and herbal teas. For example, some compositions comprise mixtures of chocolate teas and traditional teas.

### Additional Ingredients

The compositions may further include one or more additional ingredients to enhance their benefits:
Creatine: Known for improving physical performance and energy levels.
Rhodiola rosea extract: An adaptogen that may aid in stress reduction and mental clarity.
Ashwagandha extract: Another adaptogen, potentially reducing anxiety and improving focus.
Taurine: An amino acid that can support energy levels and mental focus.
Lutein: A xanthophyll carotenoid that can protect retinal tissue from oxidative stress and photodamage.
Zeaxanthin: A xanthophyll carotenoid that can protect retinal tissue from oxidative stress and photodamage.
L-theanine: An amino acid that can promote relaxation and calmness without drowsiness, and/or improve focus.
B vitamins: Essential for metabolic processes, potentially enhancing energy production.
Sweeteners and flavors: Added to improve taste and palatability.

These ingredients can be incorporated into the composition to provide additional health benefits or improve consumer acceptance.

### Methods of Production

Methods for producing the paraxanthine-containing tea compositions involve adding paraxanthine to a tea base. The tea base can be selected from decaffeinated traditional tea, non-decaffeinated traditional tea, or herbal tea.

Decaffeination. For traditional teas, decaffeination can be performed using known methods such as water extraction, solvent extraction (e.g., ethyl acetate, methylene chloride), or supercritical fluid extraction with carbon dioxide to remove at least 90% of the caffeine content.

Adding Paraxanthine. Paraxanthine can be added using various methods. Solid-solid grinding: Paraxanthine is ground with the tea material (leaves or powder) to achieve a uniform distribution, typically achieving final concentrations from 0.1% to 5% w/w. Absorption: Tea material is soaked in a paraxanthine solution (1-300 g/L) at temperatures ranging from 25° C. to 100° C. to allow absorption, followed by drying if necessary. Adsorption: Paraxanthine is applied to the surface of the tea material and fixed through drying or other means, ensuring even distribution. Mixing: Paraxanthine is directly mixed with tea, tea powder, or tea extract.

For beverage forms, paraxanthine can be added directly to the brewed tea or included in a dry mix intended to be dissolved in water, with concentrations adjusted to achieve the desired concentration in the final beverage.

Quality Control. The amount of paraxanthine in the composition can be determined using standard analytical techniques such as high-performance liquid chromatography (HPLC) or gas chromatography-mass spectrometry (GC-MS) to ensure consistency and compliance with claimed ranges.

### EXAMPLES

It is to be understood that the below-described examples are illustrative and not restrictive. The invention may be implemented in various forms without departing from the spirit or scope of the appended claims, including variations in tea type, paraxanthine concentration, and additional ingredients to meet specific consumer needs.

### Example 1: Paraxanthine-Containing Decaffeinated Black Tea

Decaffeinated black tea leaves (100 g) are mixed with paraxanthine (1 g) and ground together to achieve a paraxanthine content of 1% w/w. This composition can be used for brewing or packaged as tea leaves.

### Example 2: Paraxanthine-Containing Chrysanthemum Tea

Chrysanthemum flowers are ground into a fine powder (100 g), and paraxanthine (0.5 g) is added and uniformly blended to achieve 0.5% w/w paraxanthine. This powder can be used to prepare a herbal tea beverage.

### Example 3: Paraxanthine-Containing Matcha Tea (Decaffeinated)

Decaffeinated matcha tea powder (100 g) is mixed with paraxanthine (2 g) to achieve 2.0% w/w paraxanthine. This can be consumed as a powdered tea or mixed with water to form a beverage.

### Example 4: Paraxanthine-Containing Matcha Tea (non-decaffeinated) with Creatine

Non-decaffeinated matcha tea powder (100 g) is mixed with paraxanthine (1.5 g) to achieve 1.5% w/w paraxanthine, along with 0.5 g of creatine and 0.1 g of a sweetener (stevia). This composition provides both caffeine and paraxanthine for enhanced stimulant effects, with improved taste.

### Example 5: Paraxanthine-Containing Green Tea Beverage with Multiple Additives

A tea beverage is prepared by brewing 5 g of decaffeinated green tea leaves containing 1.0% w/w paraxanthine in 250 ml of water. To this beverage, 1 gram of taurine, 50 mg of B vitamins, and a suitable amount of sweetener are added, resulting in a final paraxanthine concentration of approximately 400 mg/L.

### Example 6: Paraxanthine-Containing Butterfly Pea Flower Tea

Butterfly pea flowers are ground into a fine powder (100 g), and paraxanthine (0.5 g) is added and uniformly blended to achieve 0.5% w/w paraxanthine. This powder can be used to prepare a herbal tea beverage.

### Example 7: Paraxanthine-Containing Chicory Chocolate Tea with Additional Ingredients

Chicory root extract (40 g) is combined with cocoa powder (20 g) and decaffeinated spray dried black tea powder (15 g) and supplemented with paraxanthine (6 g), microencapsulated lutein (3 g), salt (6 g), natural chocolate flavor (10 g), and sweetener (stevia) to create 30 servings. Each serving provides 0.2 g of paraxanthine. This mixture can be used to prepare a tea beverage.

### Example 8: Paraxanthine-Containing Fruit-flavored Decaffeinated Black Tea With L-theanine

Decaffeinated, spray-dried black tea powder (30 g) is blended with paraxanthine (6 g), L-theanine (6 g), and natural peach flavor (10 g) to create 30 servings. Each serving provides 0.2 g of paraxanthine. This powder can be dissolved in water to prepare a tea beverage.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

1. A tea composition comprising paraxanthine, or a salt or hydrate thereof, and a tea, wherein the tea composition is in a solid form or a liquid form.

2. The tea composition of claim 1, wherein the tea comprises an herbal tea.

3. The tea composition of claim 2 wherein the tea comprises one or more of chrysanthemum tea, Kenya purple leaf tea, butterfly pea flower tea, peppermint, lemongrass, and chicory root.

4. (canceled)

5. The tea composition of claim 1, wherein the tea comprises Camellia sinensis.

6. The tea composition of claim 1, wherein the tea comprises matcha tea, preferably wherein the tea composition is milled.

7. The tea composition of claim 5, wherein the tea is decaffeinated.

8. The tea composition of claim 5, wherein the tea is non-decaffeinated.

9. The tea composition of claim 1, wherein the tea comprises Theobroma caca (e.g., chocolate tea).

10. The tea composition of claim 1, wherein the tea composition comprises at least one of creatine, Rhodiola rosea extract, Ashwagandha extract, taurine, lutein, zeaxanthin, L-theanine, or one or more B vitamins.

11-18. (canceled)

19. The tea composition of claim 1, wherein the tea composition comprises one or more sweeteners and/or one or more flavors.

20. (canceled)

21. The tea composition of claim 1, wherein the tea composition is in the form of tea leaves, tea powder, or a tea beverage.

22. The tea composition of claim 1, wherein the tea composition is in solid form (e.g., tea leaves or tea powder) and 0.005% to 10% by weight of the composition is paraxanthine.

23. The tea composition of claim 22, wherein tea composition comprises about 0.1% to about 5% paraxanthine by weight.

24. The tea composition of claim 22, wherein the tea composition is freeze-dried.

25. (canceled)

26. The tea composition of claim 22, wherein the tea composition is intended to be dissolved in water to form a tea beverage.

27. The tea composition of claim 1, wherein the tea composition is in liquid form (e.g., a tea beverage), and the composition comprises 1 to 2000 mg/L paraxanthine.

28. The tea composition of claim 27, wherein tea composition comprises about 100 to about 700 mg/L paraxanthine.

29. The tea composition of claim 1, wherein the tea composition comprises about 0.2 g to about 2 g of paraxanthine per serving, preferably wherein the tea composition comprises about 0.2g to about 0.5 g of paraxanthine per serving.

30. (canceled)

31. A method of producing the tea composition of claim 1, comprising adding paraxanthine to a tea base comprising at least one of chrysanthemum tea, butterfly pea flower tea, Kenya purple leaf tea, decaffeinated tea from Camellia sinensis, tea from Theobroma caca, or matcha tea.

32. The method of claim 31, wherein paraxanthine is ground with the tea base to achieve a uniform distribution.
